(19) United States
(12) Patent Application Publication
(10) Pub. No.: US 2016/0362369 A1
(43) Pub. Date: Dec. 15, 2016

(54) METHOD FOR PRODUCING OPTICALLY ACTIVE AZETIDINONE COMPOUND

(71) Applicant: NISSAN CHEMICAL INDUSTRIES, LTD., Tokyo (JP)

(72) Inventors: Shinya NAITOU, Funabashi-shi (JP); Hirohide KITSUYAMA, Funabashi-shi (JP); Shigeo NAKAMURA, Chiyoda-ku (JP)

(73) Assignee: NISSAN CHEMICAL INDUSTRIES, LTD., Tokyo (JP)

(21) Appl. No.: 15/120,842

(22) PCT Filed: Feb. 27, 2015
(86) PCT No.: PCT/JP2015/055924

§ 371 Date: Aug. 23, 2016

(30) Foreign Application Priority Data
Mar. 6, 2014 (JP) 2014-043966
Jul. 18, 2014 (JP) 2014-147387
Feb. 6, 2015 (JP) 2015-022124

(51) Int. Cl. C07D 205/08; C07F 7/18

(52) U.S. Cl. CPC C07D 205/08; C07F 7/1844

(57) ABSTRACT

There is provided a novel and stereoselective method for producing an optically active azetidinone compound. An optically active azetidinone compound of Formula (7) can be produced in a high yield and a high stereoselectivity from a ketone compound of Formula (5), and this method is industrially useful.

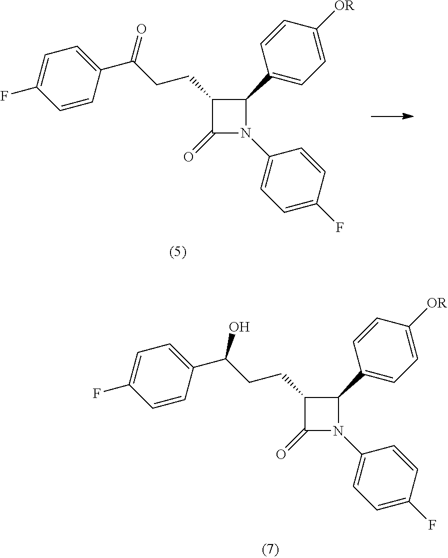

In formulae, R is a hydrogen atom or a hydroxy protecting group.

### TECHNICAL FIELD

The present invention provides to a method for producing an optically active azetidinone compound.

### BACKGROUND ART

An azetidinone compound of Formula (1) (Compound (1)) is useful as a remedy for hyperlipemia (see Non-Patent Document 1).

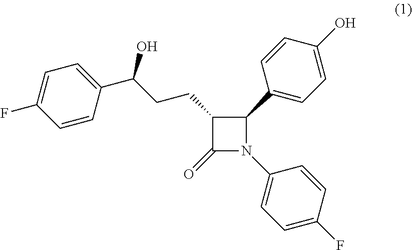 [SMILES: O=C1[C@H](CC[C@H](O)c2ccc(F)cc2)[C@@H](c2ccc(O)cc2)N1c1ccc(F)cc1]

An azetidinone compound of Formula (4) (Compound (4)) that is a precursor of Compound (1) is obtained by reducing a ketone compound of Formula (2) (Compound (2)), and, as a method for producing the azetidinone compound (Compound (4)) in high yields and with high stereoselectivity, a method for reducing the ketone compound in the presence of an asymmetric reduction catalyst such as RuCl₂((R)-xylBinap)((R)-daipen) of Formula (3) has been reported (see Patent Document 1).

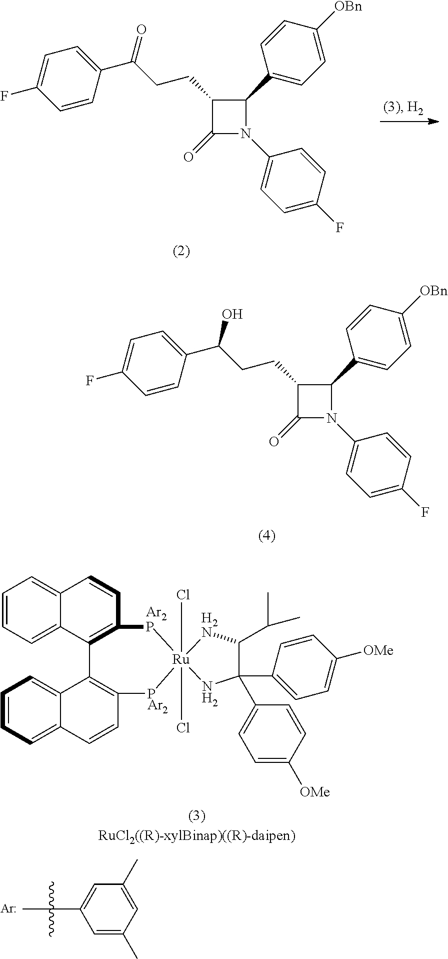

### PRIOR ART DOCUMENTS

### Patent Document

Patent Document 1: Chinese Patent Application Publication No. 102952055 (CN 102952055 A).

### Non-Patent Document

Non-Patent Document 1: Journal of Medicinal Chemistry, 1998, Vol. 41, pp. 973-980

### SUMMARY OF THE INVENTION

### Problem to be Solved by the Invention

Patent Document 1 describes that a reduction in the amount of a catalyst causes a decrease in reaction conversion and a longer reaction time. Furthermore, Patent Document 1 describes that the hydrogen pressure applied at the time of reaction is 2 to 3 MPa, and such a condition requires special equipment. Therefore, a production method improved to be industrially suitable has been desired. The present invention thus provides a method for further producing a precursor for the production of Compound (1), and Compound (1).

### Means for Solving the Problem

In view of the above-described situations, the present inventors aim to provide an industrially useful method for producing an optically active azetidinone compound in high yields and with high stereoselectivity, and, as a result of intensive study, the inventors have found a method capable of producing an optically active azetidinone compound with extremely high stereoselectivity and in high yields, and thus accomplished the present invention.

Specifically, the present invention relates to [1] to [10] below.

[1]

A method for producing an optically active azetidinone compound of Formula (7):

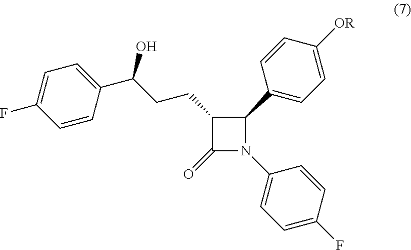

the method being characterized by reacting a ketone compound of Formula (5):

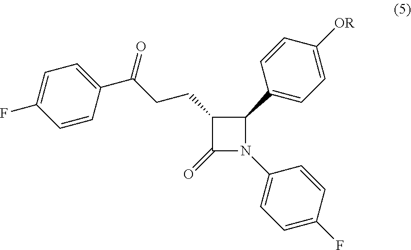

(where R is a hydrogen atom or a hydroxy protecting group) with hydrogen gas in presence of an optically active ruthenium catalyst of Formula (6):

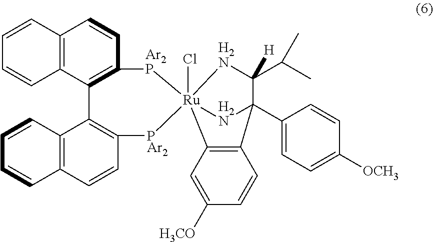

(where Ar is a 3,5-dimethylphenyl group).

[2]

The method for producing an optically active azetidinone compound according to [1], wherein R is a hydrogen atom.

[3]

The method for producing an optically active azetidinone compound according to [1], wherein R is a hydroxy protecting group.

[4]

The method for producing an optically active azetidinone compound according to [3], wherein R is a benzyl group.

[5]

The method for producing an optically active azetidinone compound according to [3], wherein R is a t-butyldimethylsilyl group.

[6]

The method for producing an optically active azetidinone compound according to [3], wherein R is an allyl group.

[7]

A method for producing an optically active azetidinone compound of Formula (1):

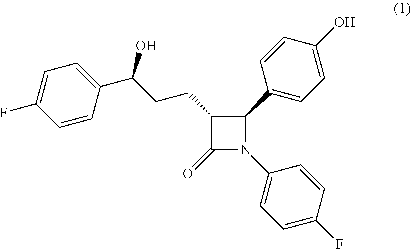 [SMILES: O=C1[C@H](CC[C@H](O)c2ccc(F)cc2)[C@@H](c2ccc(O)cc2)N1c1ccc(F)cc1]

the method being characterized by comprising:

reacting a ketone compound of Formula (5):

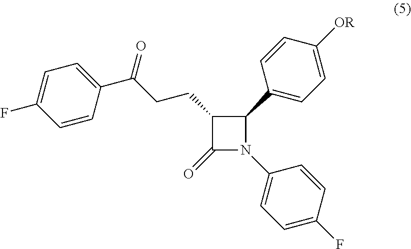

(where R is a hydroxy protecting group) with hydrogen gas in presence of an optically active ruthenium catalyst of Formula (6):

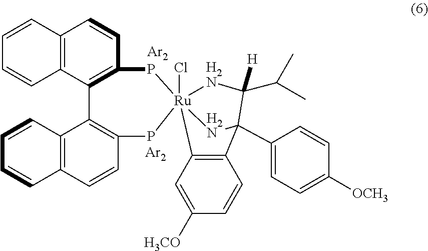

(where Ar is a 3,5-dimethylphenyl group) to induce an optically active azetidinone compound of Formula (7):

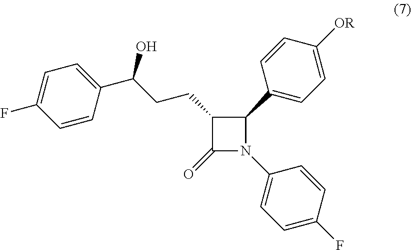

and performing a deprotection reaction.

[8]

The method for producing an optically active azetidinone compound according to [7], wherein R is a benzyl group.

[9]

The method for producing an optically active azetidinone compound according to [7], wherein R is a t-butyldimethylsilyl group.

[10]

The method for producing an optically active azetidinone compound according to [7], wherein R is an allyl group.

### Effects of the Invention

According to the present invention, the optically active azetidinone compound of Formula (7), which is useful as a pharmaceutical intermediate, can be produced in large amounts with high stereoselectivity and in high yields, and the present invention is thus of utility value as an industrial production method.

### MODES FOR CARRYING OUT THE INVENTION

The present invention will be described in more detail below.

Note that, in the present specification, “n-” denotes normal; “t-” tertiary; “c-” cyclo; “o-” ortho; “Bn” benzyl; and “TBS” t-butyldimethylsilyl.

Methods for producing optically active azetidinone compounds (7) and (1) will be described in detail below.

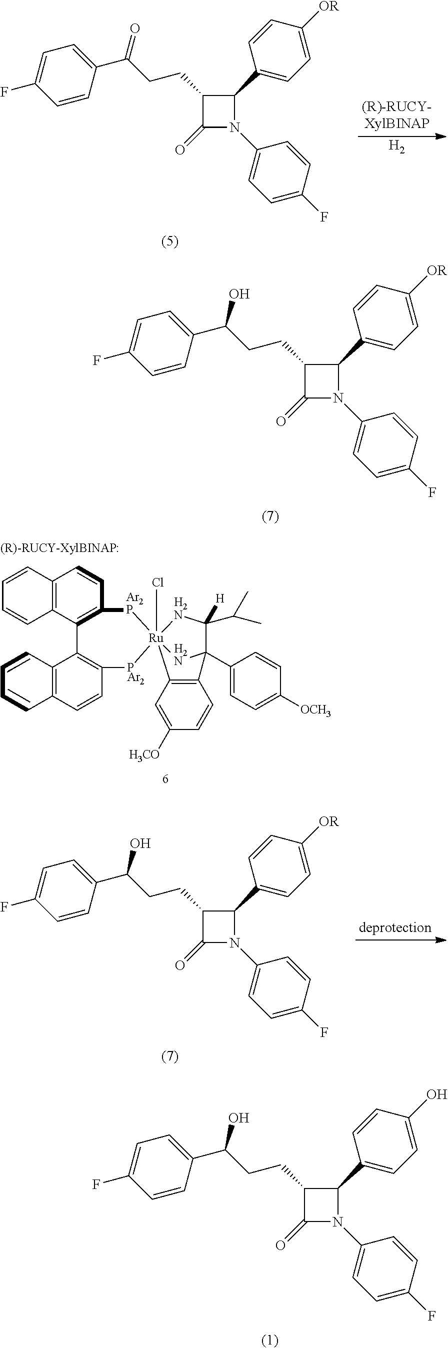

An optically active azetidinone compound (7) (where R is the same as above) can be synthesized by reacting a ketone compound (5) (where R is the same as above) with hydrogen gas in the presence of commercially available RUCY (registered trademark)-XylBINAP (manufactured by TAKASAGO INTERNATIONAL CORPORATION) of Formula (6) (where Ar is a 3,5-dimethylphenyl group).

The amount of the asymmetric reduction catalyst to be used is 1/100,000 mol % to 100 mol %, preferably 0.01 mol % to 5 mol % to the amount of the ketone compound (5).

In the production method of the present invention, a base may be added to accelerate the reaction. Preferable examples of the base to be used include an alkali metal salt of alcohol. More preferable bases are sodium methoxide, sodium ethoxide, and potassium t-butoxide, and the most preferable base is potassium t-butoxide.

The amount of the base to be added needs to be equal to or more than the amount of the asymmetric reduction catalyst to be used, preferably 10 to 500 equivalents to the catalyst.

A solvent used for the present reaction is not limited unless the solvent inhibits the reaction from proceeding, and preferable examples of the solvent include water, aprotic polar organic solvents (such as N,N-dimethylformamide, dimethyl sulfoxide, N,N-dimethylacetamide, tetramethyl urea, sulfolane, N-methylpyrrolidone, and N,N-dimethylimidazolidinone), ether solvents (such as diethyl ether, diisopropyl ether, t-butylmethyl ether, tetrahydrofuran, and dioxane), aliphatic hydrocarbon solvents (such as pentane, n-hexane, c-hexane, octane, decane, decalin, and petroleum ether), aromatic hydrocarbon solvents (such as benzene, chlorobenzene, o-dichlorobenzene, nitrobenzene, toluene, xylene, mesitylene, and tetralin), halogenated hydrocarbon solvents (such as chloroform, dichloromethane, dichloroethane, and carbon tetrachloride), lower fatty acid ester solvents (such as methyl acetate, ethyl acetate, butyl acetate, and methyl propionate), alkoxy alkane solvents (such as dimethoxyethane and diethoxyethane), alcohol solvents (such as methanol, ethanol, 1-propanol, and 2-propanol), and carboxylic acid solvents (such as acetic acid). Among them, alcohol solvents are preferable. The alcohol solvents may be used singly, or in combination as a mixture of two or more of them.

The reaction temperature is preferably about 10° C. to 50° C., more preferably about 20° C. to 30° C.

A reducing agent that may be used is not particularly limited as long as the reducing agent has been used as a reagent, and examples of the reducing agent include hydrogen gas.

The hydrogen pressure for the reaction may be arbitrary, but, is preferably in a range of 0.1 MPa to 4.0 MPa, more preferably in a range of 0.1 MPa to 1.0 MPa from viewpoints of contribution to the reaction and conformity to industrial production.

A hydroxy protecting group can be removed by performing a deprotection reaction (see, for example, Protective Groups in Organic Synthesis, Fourth edition, written by T. W. Greene, John Wiley & Sons Inc., 2006).

The hydroxy protecting group in the present production method is not limited unless the hydroxy protecting group inhibits the reaction from proceeding, and a hydroxy protecting group to be typically used in organic synthesis may be used. Preferable examples of the hydroxy protecting group include benzyl group, methyl group, methoxy methyl group, acetyl group, trimethylsilyl group, t-butyldimethylsilyl group, and allyl group, and, more preferably, benzyl group, t-butyldimethylsilyl group, and allyl group.

In the present specification, “allyl group” is a monovalent substituent of CH₂═CH—CH₂—.

The ketone compounds (2) and (8) are well-known compounds, and can be synthesized in accordance with methods described in documents, such as International Publication No. 2007/119106 (WO 2007/119106) and International Publication No. 2007/072088 (WO 2007/072088).

### EXAMPLES

Hereinafter, the present invention will be described more specifically with reference to Examples, but, the present invention is not limited to Examples below. Note that measurements of nuclear magnetic resonance spectra (¹H-NMR) and high performance liquid chromatography (HPLC) analyses were performed with the following apparatuses under the following conditions. V in an expression (V/V/V) denotes volume.

[1] ¹H-NMR

Apparatus model: ECP300 (manufactured by JEOL Ltd.)

Solvent for measurement: DMSO-d₆

[2] HPLC

(1) HPLC analysis condition 1 (used for measurement of chemical purity and reaction conversion)

Apparatus: Agilent 1260

Column: L-Column ODS (manufactured by Chemicals Evaluation and Research Institute, Japan)

250×4.6 mm I.D., 5 μm

Column oven temperature: 40° C.

Eluent: Mixed solvent of acetonitrile and 10 mM ammonium acetate aqueous solution

Gradient condition (acetonitrile volume ratio): 20% (0 min.)→20% (10 min.)→50% (15 min.)→50% (30 min.)→95% (40 min.)→95% (50 min.)

The time programs in the parentheses denote the total time from the start of analysis.

Flow rate: 1.0 mL/min.

Detector: ultraviolet-visible absorption spectrophotometer 233 nm

(2) HPLC analysis condition 2 (used for measurement of optical purity)

Apparatus: SHIMADZU 20A

Column: CHIRALPAK IC (manufactured by Daicel Corporation)

250 mm×4.6 mm I.D., 5 μm

Column oven temperature: 35° C.

Fluent: hexane/2-propanol/trifluoroacetic acid=900/100/1 (V/V/V)

Flow rate: 1.0 mL/min.

Detector: ultraviolet-visible absorption spectrophotometer 233 nm

### Example 1

### Synthesis of Compound (1)

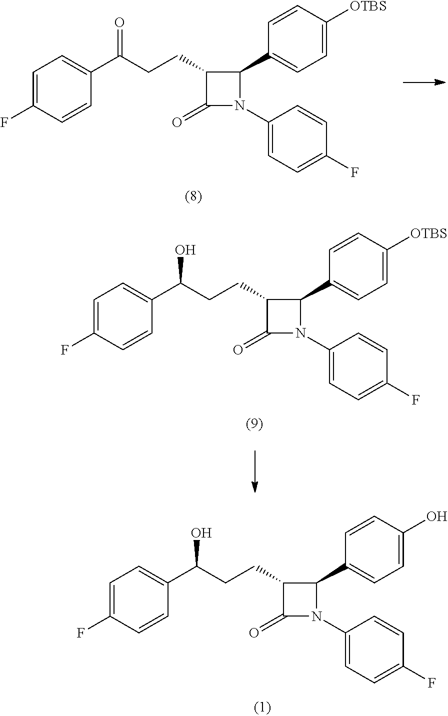 [SMILES: CC(C)(C)[Si](C)(C)Oc1ccc([C@@H]2[C@@H](CCC(=O)c3ccc(F)cc3)C(=O)N2c2ccc(F)cc2)cc1 ; CC(C)(C)[Si](C)(C)Oc1ccc([C@@H]2[C@@H](CC[C@H](O)c3ccc(F)cc3)C(=O)N2c2ccc(F)cc2)cc1 ; O=C1[C@H](CC[C@H](O)c2ccc(F)cc2)[C@@H](c2ccc(O)cc2)N1c1ccc(F)cc1]

2.09 g (4.01 mmol) of Compound (8) synthesized in accordance with the method described in International Publication No. 2007/072088 (WO 2007/072088) was dissolved in 20.91 g of ethanol, and 22.4 mg (0.20 mmol) of potassium t-butoxide and 4.7 mg (0.004 mmol, 0.1 mol % to Compound (8)) of (R)-RUCY (registered trademark)-XylBINAP (manufactured by TAKASAGO INTERNATIONAL CORPORATION) were added and dissolved. After the reactor was purged with hydrogen gas, the mixture was stirred at a hydrogen pressure of 500 kPa and at a reaction temperature of 26° C. for 3 hours. Then, 15.0 mg (0.25 mmol) of acetic acid was added to the mixture, and the solvent was distilled off under reduced pressure to obtain 3.81 g of a residue. The residue was dissolved in 10.48 g of ethyl acetate, and 0.10 g of activated carbon (Special SHIRASAGI, manufactured by Japan Enviro Chemicals, Limited) was added to the solution, followed by stirring for 40 minutes at room temperature. The activated carbon was filtered off, and washed with 2.1 g of ethyl acetate. The filtrate and washings were mixed, and the solvent was distilled off under reduced pressure to obtain 3.28 g of a residue. The residue was dissolved in 20.92 g of 2-propanol, and again the solvent was distilled off under reduced pressure, whereby the total amount of solution was 10.56 g and a 2-propanol solution of Compound (9) was thus obtained.

1.37 g (2.79 mmol) of a 20% sulfuric acid aqueous solution was added to the 2-propanol solution, and the mixture was stirred at 58° C. to 59° C. for one hour, and then allowed to cool, whereby a white solid was precipitated. To the resultant solution, 8.46 g of water was added dropwise, followed by further stirring at 0° C. to 3° C. for 3 hours, and the precipitated solid was filtered off. The solid was washed with a mixed solution of 2.09 g of 2-propanol and 2.09 g of water, a mixed solution of 2.11 g of 2-propanol and 2.10 g of water, and 2.11 g of water, and dried under reduced pressure at 50° C. to obtain 1.34 g of Compound (1) as a white solid.

Under HPLC analysis condition 1, the chemical purity of the obtained Compound (1) was measured to be 97.49%.

Under HPLC analysis condition 2, the diastereomer excess was measured to be 99.01% de. Furthermore, the retention time coincided with the retention time of Compound (1) synthesized in accordance with a method described in Japanese Patent No. 3640888 (JP 3640888) (the retention time of Compound (1): 19.7 minutes, the retention time of the diastereomer: 24.6 minutes).

Furthermore, the diastereomer excess of (1), which is a two-step-reaction product, was 99.01% de, and the diastereomer excess is considered to be retained at the second step, and it is therefore understood that the diastereomer excess of Compound (9), that is, a product of the first step reaction, was 99.01% de or higher.

¹H-NMR analysis: (300 MHz, DMSO-d₆) δ ppm: 1.72-1.88 (4H, m), 3.06-3.08 (1H, m), 4.49 (1H, s), 4.80 (1H, d, J=2.2 Hz), 5.28 (1H, br), 6.76 (2H, d, J=8.5 Hz), 7.09-7.16 (4H, m), 7.19-7.24 (4H, m), 7.28-7.33 (2H, m), 9.53 (1H, s)

### Example 2

### Synthesis of Compound (4)

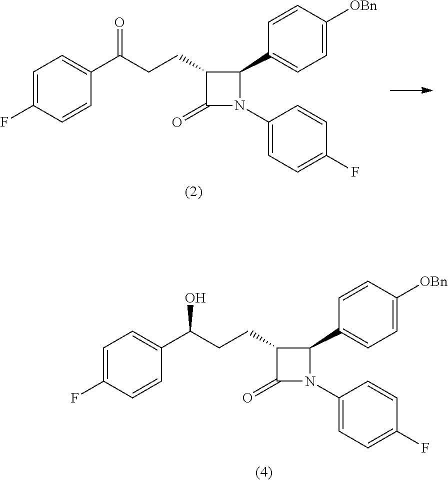 [SMILES: O=C(CC[C@H]1C(=O)N(c2ccc(F)cc2)[C@@H]1c1ccc(OCc2ccccc2)cc1)c1ccc(F)cc1 ; O=C1[C@H](CC[C@H](O)c2ccc(F)cc2)[C@@H](c2ccc(OCc3ccccc3)cc2)N1c1ccc(F)cc1]

54.00 g (108.1 mmol) of Compound (2) synthesized in accordance with a method described in Japanese Unexamined Patent Application Publication (Translation of PCT Application) No. H08-509989 (JP H08-509989 A) was dissolved in 540.19 g of ethanol and 270.20 g of tetrahydrofuran, and 587.3 mg (5.23 mmol) of potassium t-butoxide and 127.5 mg (0.1076 mmol, 0.1 mol % to Compound (2)) of (R)-RUCY (registered trademark)-XylBINAP (manufactured by TAKASAGO INTERNATIONAL CORPORATION) were added and dissolved. After the reactor was purged with hydrogen gas, the mixture was stirred at a hydrogen pressure of 500 kPa and a temperature of 11° C. to 21° C. for 6 hours. The conversion of Compound (2) into Compound (4) was 100.00%. To the obtained solution, 423.7 mg (7.06 mmol) of acetic acid was added and stirred, and then, insoluble matter was filtered off. The obtained filtrate was concentrated to 104.78 g under reduced pressure, and then 327.82 g of ethanol was added to the filtrate, followed by heating to 71° C. The heated mixture was cooled to 22° C., and then 54.00 g of water was added, followed by cooling to 1° C. A precipitated solid was filtered off, and washed with a mixed solution of 43.23 g of ethanol and 10.94 g of water, and further washed with a mixed solution of 43.28 g of ethanol and 10.82 g of ion exchange water, whereby 49.73 g of Compound (4) was obtained as a white solid. The chemical purity of the obtained Compound (4) was 99.96%, and the diastereomer excess was 99.88% de.

¹H-NMR analysis: (300 MHz, CDCl₃) δ ppm: 1.84-1.99 (4H, m), 2.38 (1H, d, J=2.9 Hz), 3.00 (1H, d, J=5.2 Hz), 4.56 (1H, br), 4.64 (1H, br), 5.04 (2H, s), 6.88-7.03 (6H, m), 7.20-7.42 (1H, m)

### Example 3

### Synthesis of Compound (4)

2.00 g (4.00 mmol) of Compound (2) obtained in the same manner as in Example 2 and 22.4 mg (0.20 mmol) of potassium t-butoxide were suspended in 29 mL of ethanol, and then 1 ml (0.81 μmol, 0.02 mol % to Compound (2)) of a solution prepared by dissolving 9.6 mg (0.0081 mmol) of (R)-RUCY (registered trademark)-XylBINAP (manufactured by TAKASAGO INTERNATIONAL CORPORATION) in 10 mL of ethanol was added to the suspension. After the reactor was purged with hydrogen gas, the mixture was stirred at a hydrogen pressure of 2000 kPa and at a reaction temperature of 44° C. for 1 hour. The conversion of Compound (2) into Compound (4) was 99.21%, and the diastereomer excess was 99.78% de. The solution was concentrated under reduced pressure to obtain 1.99 g of Compound (4) as a white solid.

### Example 4

### Synthesis of Compound (4)

1.89 g of Compound (4) was obtained in the same method as in Example 3, except that the hydrogen pressure in Example 3 was changed to 500 kPa.

The conversion of Compound (2) into Compound (4) one hour after the stirring was 97.44%, and the diastereomer excess was 100.00% de.

### Example 5

### Synthesis of Compound (1)

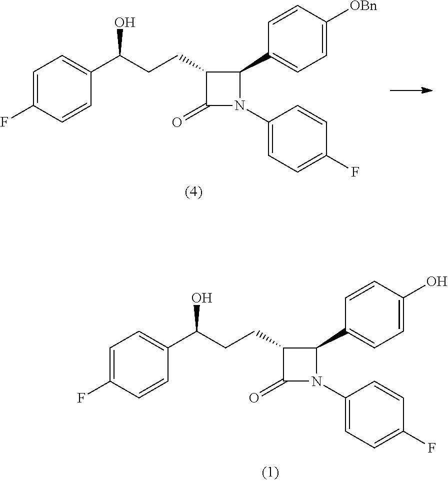 [SMILES: O=C1[C@H](CC[C@H](O)c2ccc(F)cc2)[C@@H](c2ccc(OCc3ccccc3)cc2)N1c1ccc(F)cc1 ; O=C1[C@H](CC[C@H](O)c2ccc(F)cc2)[C@@H](c2ccc(O)cc2)N1c1ccc(F)cc1]

13.00 g (26.02 mmol) of Compound (4) synthesized in accordance with Example 2 was dissolved in 78.12 g of ethyl acetate and 37.70 g of methanol, and then 3.13 g (52.12 mmol) of acetic acid and 1.42 g of palladium on carbon (54.10% of moisture) were added. To the resultant solution, 27.35 g (26.02 mmol) of a 6% ammonium formate methanol solution prepared from 6.00 g of ammonium formate and 94.00 g of methanol was added dropwise, followed by stirring. Furthermore, 3.01 g (2.86 mmol) of the 6% ammonium formate methanol solution was added 6.5 hours later; 1.97 g (1.87 mmol) 9 hours later; and 1.91 g (1.82 mmol) 10 hours later, and the reaction was completed. Celite filtration was then performed to remove palladium on carbon. Celite was washed with 13.11 g, 12.96 g, and 13.06 g of ethyl acetate, and the solvent was distilled off under reduced pressure, so that the solution was concentrated to 37.79 g, and then 104.06 g of ethyl acetate was added to the solution. The solvent was distilled off under reduced pressure so that the solution was concentrated to 78.15 g, and then 52.21 g of a 5% sodium bicarbonate aqueous solution was added to perform liquid-liquid extraction. 39.03 g of water was added to an obtained organic phase to perform liquid-liquid extraction again, and the resultant organic phase was filtered, and then the solvent was distilled under reduced pressure, so that the solution was concentrated to 45.60 g. 5.07 g of heptane was added dropwise to the solution at 38° C., followed by stirring, whereby a solid was precipitated. Furthermore, 25.35 g of heptane was added dropwise to the solution, followed by cooling to −1° C., and the resultant solution was filtered to obtain the solid. The obtained solid was washed with a mixed solution of 4.82 g of ethyl acetate and 9.65 g of heptane, and dried under reduced pressure at 50° C. to obtain 9.79 g of Compound (1) as a white solid. The chemical purity of the obtained Compound (1) was 99.82%. Under HPLC analysis condition 2, the diastereomer excess was measured to be 99.78% de. Furthermore, the retention time coincided with the retention time of Compound (1) synthesized in accordance with the method described in Japanese Patent No. 3640888 (JP 3640888) (the retention time of Compound (1): 20.6 minutes, the retention time of the diastereomer: 26.1 minutes).

The XRD of this white solid was measured and found that the XRD coincided with the diffraction pattern of Form A described in Japanese Unexamined Patent Application Publication (Translation of PCT Application) No. 2007-526251 (JP 2007-526251 A).

### Example 6

### Synthesis of Compound (11)

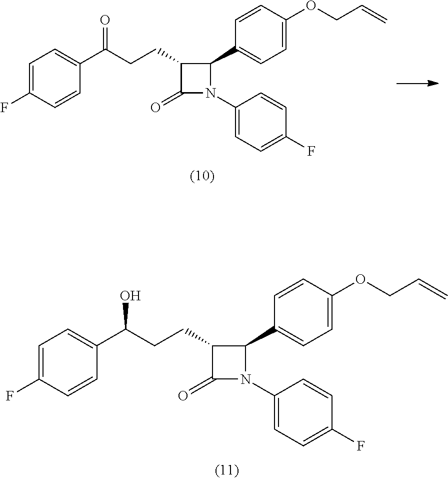 [SMILES: C=CCOc1ccc([C@@H]2[C@@H](CCC(=O)c3ccc(F)cc3)C(=O)N2c2ccc(F)cc2)cc1 ; C=CCOc1ccc([C@@H]2[C@@H](CC[C@H](O)c3ccc(F)cc3)C(=O)N2c2ccc(F)cc2)cc1]

5.3 mg (0.0045 mmol) of (R)-RUCY (registered trademark)-XylBINAP (manufactured by TAKASAGO INTERNATIONAL CORPORATION) and 10.04 g of ethanol were added to 2.00 g (4.47 mmol) of Compound (10) synthesized in accordance with a method described in International Publication No. 2009/157019 (WO 2009/157019), and suspended. Then, 34.0 mg (0.2235 mmol) of diazabicycloundecene was added to the suspension. After the reactor was purged with hydrogen gas, the mixture was stirred at a hydrogen pressure of 500 kPa and at a reaction temperature of 20° C. for 7 hours. To the obtained solution, 33.5 mg (0.5587 mmol) of acetic acid was added and stirred, and then the solvent was distilled off under reduced pressure to obtain 1.98 g of Compound (11) as a light brown solid. The chemical purity of the obtained compound (11) was 96.50%, and the diastereomer excess was 99.87% de.

¹H-NMR analysis: (300 MHz, CDCl₃) 6 ppm: 1.85-2.02 (4H, m), 2.24 (1H, d, J=3.8 Hz), 3.05-3.10 (1H, m), 4.51-4.54 (2H, m), 4.57 (2H, d, J=16.7 Hz), 4.71-4.73 (1H, m), 5.27-5.44 (2H, m), 5.98-6.10 (1H, m), 6.89-7.05 (6H, m), 7.19-7.31 (6H, m)

### Example 7

### Synthesis of Compound (1)

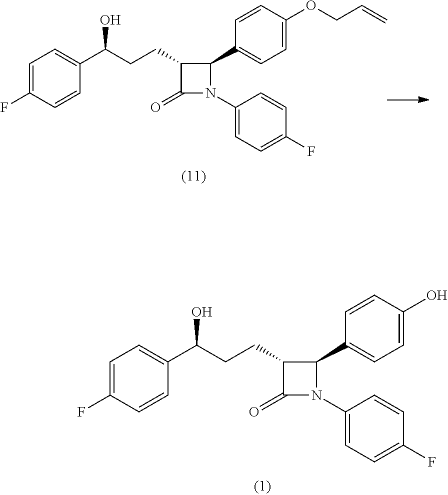 [SMILES: C=CCOc1ccc([C@@H]2[C@@H](CC[C@H](O)c3ccc(F)cc3)C(=O)N2c2ccc(F)cc2)cc1 ; O=C1[C@H](CC[C@H](O)c2ccc(F)cc2)[C@@H](c2ccc(O)cc2)N1c1ccc(F)cc1]

1.97 g (4.38 mmol) of Compound (11) synthesized in Example 6 was dissolved in 20.06 g of ethyl acetate, and then 411.5 mg (8.94 mmol) of formic acid, 583.7 mg (6.70 mmol) of morpholine, and 31.3 mg (0.0268 mmol) of tetrakis(triphenylphosphine)palladium were added, followed by stirring at 55° C. to 60° C. for 50 minutes. The solution was cooled to 24° C., and then 6.02 g of 1M hydrochloric acid was added to perform liquid-liquid extraction. To 20.36 g of an obtained organic phase, 4.02 g of water was added to perform liquid-liquid extraction, whereby 19.55 g of an organic phase was obtained. The solvent was distilled off under reduced pressure, so that the solution was reduced to 3.43 g, and then 20.28 g of 2-propanol was added to the solution. The solvent was distilled off under reduced pressure, so that the solution was reduced to 3.65 g, and then 2-propanol was added to provide a solution of 5.22 g. 1.75 g of water was added to the solution at 55° C. to 56° C. and stirred, whereby a solid was precipitated. The solution was cooled to 1° C. and filtrated to obtain the solid, and the solid was washed with a mixed solution of 0.88 g of 2-propanol and 0.89 g of water, and dried under reduced pressure at 50° C. to obtain 1.48 g of Compound (1) as a light yellow solid. The chemical purity of the obtained compound was 96.93%. The diastereomer excess was 100.00% de. Furthermore, the retention time coincided with the retention time of Compound (1) synthesized in accordance with the method described in Japanese Patent No. 3640888 (JP 3640888) (the retention time of Compound (1): 20.1 minutes, the retention time of the diastereomer: 25.0 minutes).

### INDUSTRIAL APPLICABILITY

The present invention is useful in that the invention is capable of producing an optically active azetidinone compound useful as a pharmaceutical intermediate in high yields and with high stereoselectivity.

1. A method for producing an optically active azetidinone compound of Formula (7): 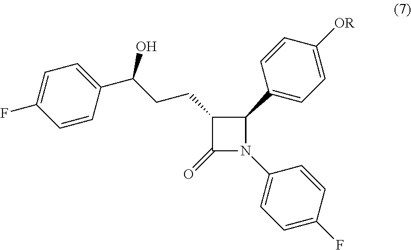
the method being characterized by reacting a ketone compound of Formula (5): 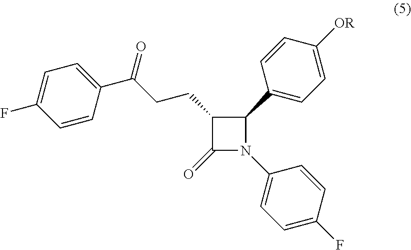
(where R is a hydrogen atom or a hydroxy protecting group) with hydrogen gas in presence of an optically active ruthenium catalyst of Formula (6): 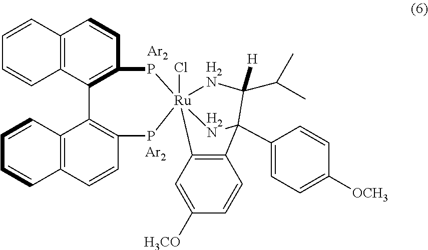
(where Ar is a 3,5-dimethylphenyl group).

2. The method for producing an optically active azetidinone compound according to claim 1, wherein R is a hydrogen atom.

3. The method for producing an optically active azetidinone compound according to claim 1, wherein R is a hydroxy protecting group.

4. The method for producing an optically active azetidinone compound according to claim 3, wherein R is a benzyl group.

5. The method for producing an optically active azetidinone compound according to claim 3, wherein R is a t-butyldimethylsilyl group.

6. The method for producing an optically active azetidinone compound according to claim 3, wherein R is an allyl group.

7. A method for producing an optically active azetidinone compound of Formula (1): 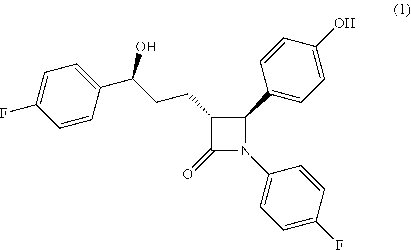 [SMILES: O=C1[C@H](CC[C@H](O)c2ccc(F)cc2)[C@@H](c2ccc(O)cc2)N1c1ccc(F)cc1]
the method being characterized by comprising:
reacting a ketone compound of Formula (5): 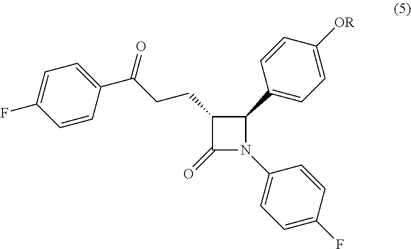
(where R is a hydroxy protecting group) with hydrogen gas in presence of an optically active ruthenium catalyst of Formula (6): 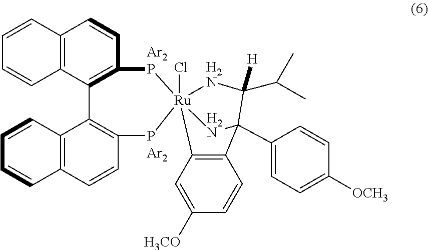
(where Ar is a 3,5-dimethylphenyl group) to induce an optically active azetidinone compound of Formula (7): 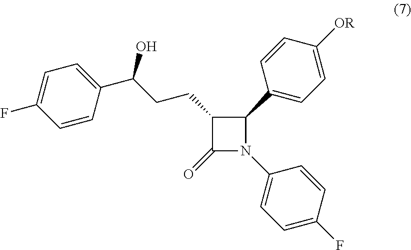
and performing a deprotection reaction.

8. The method for producing an optically active azetidinone compound according to claim 7, wherein R is a benzyl group.

9. The method for producing an optically active azetidinone compound according to claim 7, wherein R is a t-butyldimethylsilyl group.

10. The method for producing an optically active azetidinone compound according to claim 7, wherein R is an allyl group.
